SEQUENCE LISTING (8 sequences)

SEQ ID NO: 1 - DNA, 36 bp, synthetic construct
  source 1..36: mol_type = other DNA
nnnnnnnntctcgcgcagatcggaagagcgtcgtgt

SEQ ID NO: 2 - DNA, 30 bp, synthetic construct
  source 1..30: mol_type = other DNA
nnnnncgatgagatcggaagagcgtcgtgt

SEQ ID NO: 3 - DNA, 30 bp, synthetic construct
  source 1..30: mol_type = other DNA
nnnnnttaggagatcggaagagcgtcgtgt

SEQ ID NO: 4 - DNA, 30 bp, synthetic construct
  source 1..30: mol_type = other DNA
nnnnntgaccagatcggaagagcgtcgtgt

SEQ ID NO: 5 - DNA, 15 bp, synthetic construct
  source 1..15: mol_type = other DNA
acacgacgctcttcc

SEQ ID NO: 6 - DNA, 22 bp, synthetic construct
  source 1..22: mol_type = other DNA
agatcggaagagcacacgtctg

SEQ ID NO: 7 - DNA, 70 bp, synthetic construct
  source 1..70: mol_type = other DNA
aatgatacggcgaccaccgagatctacactatagcctacactctttccctacacgacgctcttccgatct

SEQ ID NO: 8 - DNA, 66 bp, synthetic construct
  source 1..66: mol_type = other DNA
caagcagaagacggcatacgagatcgagtaatgtgactggagttcagacgtgtgctcttccgatct

(19) United States
(12) Patent Application Publication
(10) Pub. No.: US 2023/0313177 A1
(43) Pub. Date: Oct. 5, 2023

(54) METHODS FOR OLIGO TARGETED PROXIMITY LIGATION

(71) Applicant: Eclipse Bioinnovations, Inc., San Diego, CA (US)

(72) Inventors: Daniel A. Lorenz, San Diego, CA (US); Karen B. Chapman, San Diego, CA (US)

(21) Appl. No.: 18/173,644

(22) Filed: Feb. 23, 2023

Related U.S. Application Data
(60) Provisional application No. 63/313,906, filed on Feb. 25, 2022.

(51) Int. Cl. C12N 15/10

(52) U.S. Cl. CPC C12N 15/1065

(57) ABSTRACT

The present disclosure relates to methods of enriching for nucleic acid sequence using a targeting proximity-base ligation. In some embodiments, the method comprises preparing nucleic acid from a sample, complexing the nucleic acid from a sample with the one or more targeting oligonucleotides comprising a targeting region linked to a reverse transcription region, and preparing a library of nucleic acids amplified from the oligonucleotides. Further, kits are disclosed for preparing and producing the methods described herein.

### CROSS-REFERENCE TO RELATED APPLICATIONS

This application claims the benefit of U.S. Provisional Application No. 63/313,906 filed on Feb. 25, 2022, which is incorporated by reference in its entirety.

### BACKGROUND

In eukaryotes, most messenger RNAs and some non-coding RNAs, such as long non-coding RNAs and primary microRNAs, undergo polyadenylation at the 3′ end of the transcripts, which serves as a mechanism for RNA stability and processing efficiency. Recent applications of transcriptome-wide techniques have revealed the presence of multiple polyadenylation sites in most eukaryotic genes, and that the biosynthesis of poly(A) tails often concurs with the selection of a particular poly(A) site in the nucleus. A 3′-poly(A) tail is typically a monotonous sequence of adenine nucleotides, which is enzymatically added by poly(A) polymerases (PAP) to the 3′-end of the nascent mRNA. The poly(A) sequence is added to the 3′-end of RNA molecules by a cell’s ubiquitous cleavage/polyadenylation machinery. After cleavage, most pre-mRNAs, with the exception of replication-dependent histone transcripts, acquire a polyadenylated tail. In this context, 3′-end processing is a nuclear cotranscriptional process that promotes the transport of mRNA from the nucleus to the cytoplasm and affects the stability and the translation of mRNAs.

Formation of the 3′ polyadenylated end occurs in a two-step reaction directed by the cleavage/polyadenylation machinery and depends on the presence of two sequence elements in mRNA precursors (pre-mRNAs); a highly conserved hexanucleotide AAUAAA (polyadenylation signal) and a downstream G/U-rich sequence. In a first step, pre-mRNAs are cleaved between these two elements. In a second step, tightly coupled to the first step, the newly formed 3′ end is extended by the addition of a poly(A) sequence consisting of 200-250 adenylates which subsequently affects all aspects of mRNA metabolism, including mRNA export, stability and translation (Dominski and Marzluff, 2007, Gene 396(2): 373-90.). 5′ cap structures can also be introduced into in vitro transcribed RNA (Pascolo S., 2006, Methods Mol Med., 127:23-40.).

Typically, the poly(A) tail of a mammalian mRNA contains about 250 adenine nucleotides. It was found that the length of such a poly(A) tail may be a potentially critical element for the stability of the individual mRNA.

### REFERENCE TO SEQUENCE LISTING

The present application is filed with a Sequence Listing in Electronic format. The Sequence Listing is provided as a file entitled EBIO.006WO ST 26.xml, created Feb. 17, 2023, which is approximately 8 kb in size. The information in the electronic format of the sequence listing is incorporated herein by reference in its entirety.

### SUMMARY

In aspects, the disclosure relates to a method of oligonucleotide targeted proximity ligation. In some embodiments, the method includes preparing RNA from a sample, preparing one or more oligonucleotides, wherein the one or more oligonucleotides comprise a targeting region and a barcode region, complexing the RNA from a sample with the one or more targeting oligonucleotides, and preparing a library of nucleic acids amplified from the one or more oligonucleotides. In some embodiments, the one or more oligonucleotides includes a targeting region, an alkyl linker, and a reverse transcription region. In some embodiments, the one or more oligonucleotides includes a targeting region, a polyethylene glycol (PEG) linker, and a reverse transcription region. In some embodiments, the one or more oligonucleotides further includes one or more of a 5′ biotin, a 3′ biotin, a 5′ azide, a 3′ azide, a 5′ alkyne, a 3′ alkyne, and a 5′ phosphate. In some embodiments, preparing the RNA from a sample further includes isolating cells. In some embodiments, the method further includes measuring mRNA concentration. In some embodiments, the method further includes measuring total RNA concentration. In some embodiments, the method further includes fragmenting RNA. In some embodiments, the method further includes treating RNA with a RNase. In some embodiments, the barcode region includes one or more barcodes. In some embodiments, preparing the library includes coupling the one or more oligonucleotides to a magnetic bead. In some embodiments, preparing the library includes a precipitation step. In some embodiments, the precipitation step includes a first precipitation wash. In some embodiments, the precipitation step includes RNA end repair. In some embodiments, the precipitation step includes a second precipitation wash. In some embodiments, preparing the library further includes barcode chimeric ligation. In some embodiments, preparing the library further includes proteinase digestion of samples. In some embodiments, preparing the library further includes a clean up step and a concentration step. In some embodiments, the method further includes a reverse transcription of the RNA sample. In some embodiments, the method further includes repairing cDNA ends. In some embodiments, the method further includes a cDNA sample bead cleanup step. In some embodiments, the method further includes a cDNA sample quantification by qPCR step. In some embodiments, the method further includes PCR amplification of cDNA and dual index addition. In some embodiments, the method further includes targeting region of the one or more oligonucleotides is complementary to the polyA tail. In some embodiments, the targeting region of the oligo is complementary to a gene of interest.

In other aspects, a kit is described herein. In some embodiments, the kit comprises one or more targeted oligonucleotides, and a manual providing instructions for proximity based ligation. In some embodiments, the kit further includes one or more buffers. In some embodiments, the one or more buffers includes bead elution buffer, library elution buffer, PNK buffer, RT buffer, proteinase K buffer, bead binding buffer, RNA ligation buffer, ssDNA ligation buffer, coupling buffer, and combination thereof. In some embodiments, the kit further includes one or more primers. In some embodiments, the one or more primers is selected from the group consisting of qPCR primer and RT primer.

These and other features, aspects, and advantages of the present disclosures will become better understood with reference to the following description, drawings, and claims.

### BRIEF DESCRIPTION OF THE DRAWINGS

FIG. 1 illustrates two oligo designs for a proximity-based sequence specific ligation.

FIG. 2 illustrates a schematic diagram depicting an embodiment of a protocol for proximity-base sequence specific ligations.

FIG. 3 illustrates a genome track view of one a polyA tail enrichment using proximity-based polyT ligations and depicts 3′ coverage bias.

FIG. 4, Panel A illustrates the read structure of a polyA tail enrichment using proximity-based polyT ligations. FIG. 4, Panel B is a table of read 1 from a polyA tail enrichment and depicts sequencing of the barcode (BC), unique molecular index (UMI), and polyA tails (complementary strand) in the sequencing insert. FIG. 4, Panel C is a table of read 2 from a polyA tail enrichment and depicts sequencing of expressed genes within the sample with sequencing extending into the polyA tail.

FIG. 5 illustrates a schematic diagram depicting an embodiment of a protocol for multiplexing oligo targeting proximity-ligations with other proximity-based ligation technologies.

FIG. 6 illustrates a genome track view of a multiplexed assay diplaying only read 2. The top coverage track depicts the 5′ cap bias of a m7G antibody. The middle coverage track depicts 3′ bias of the polyT targeting proximity ligation oligo. The bottom coverage track depicts the enrichment of reads centered on m6A motifs (RRACT).

### DETAILED DESCRIPTION

In the Summary Section, and brief description of the drawings above and the Detailed Description Section, and the claims below, reference is made to particular features of the disclosure. It is to be understood that the disclosure in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Central to both DNA and RNA function is their ability to anneal to separate strands through sequence specific base pairing. Several technologies and therapies, such as polyA tail pull down, and antisense therapeutics, depend on accurate base pairing. To further capitalize on this feature, embodiments of the invention relate to methods for a proximity-based ligation capable of targeting select RNAs containing a desired sequence. In some embodiments, the proximity-based ligation products can contain barcodes. In some embodiments, the proximity-based ligation may be multiplexed for enrichment-based sequencing.

### Methods

In some aspects, the disclosure relates to a method of oligonucleotide targeted proximity ligation. In some embodiments, the method includes hybridizing a nucleic acid sample with one or more oligonucleotides, performing a proximity based ligation between the nucleic acid sample and one or more hybridized oligonucleotides, and preparing a library of nucleic acids amplified from the one or more oligonucleotides. In some embodiments, the one or more oligonucleotides include a targeting region and an amplification region. In some embodiments, the nucleic acid sample includes RNA. In some embodiments, the nucleic acid includes DNA. In some embodiments, the method may further include one or more of the steps from generating an oligo with a sequence complementary region and a reverse transcription sequence, contacting the nucleic acid sample with the oligo, ligating the barcode region of the oligo to the bound nucleic acid sample to form chimeric nucleic acid molecules, amplifying enriched chimeric nucleic acid molecules, RNA or cDNA molecules thereof, by PCR, sequencing the PCR products, and identifying computationally chimeric RNA molecules. In some embodiments, the method may be used to determine a polyA length of target genes. In some embodiments, the method may be used to determine the composition of a polyA tail of a target gene.

In some aspects, the disclosure relates to a method of oligonucleotide targeted proximity ligation. In some embodiments, the method comprises preparing RNA from a sample, preparing one or more oligonucleotides containing a targeting region and a barcode region, complexing the RNA from a sample with the one or more targeting oligonucleotides, and preparing a library of nucleic acids amplified from the oligonucleotides. In some embodiments, the method may further include one or more of the steps from generating an oligo with a sequence complementary region and a reverse transcription sequence, contacting an RNA sample with the oligo, ligating the barcode region of the oligo to the bound RNA molecule to form chimeric RNA molecules, amplifying enriched chimeric RNA molecules, or cDNA molecules thereof, by PCR, sequencing the PCR products, and identifying computationally chimeric RNA molecules. In some embodiments, the method may be used to determine the polyA length of target genes. In some embodiments, the method may be used to determine the composition of the polyA tail of a target gene.

In some embodiments, the one or more oligonucleotides includes a targeting region. In some embodiments, the one or more oligonucleotides includes an alkyl linker. In some embodiments, the one or more oligonucleotides includes a polyethylene glycol (PEG) linker. In some embodiments, the one or more oligonucleotides includes a reverse transcription region. In some embodiments, the one or more oligonucleotides includes a targeting region and an alkyl linker. In some embodiments, the one or more oligonucleotides includes a targeting region and a PEG linker. In some embodiments, the one or more oligonucleotides includes an alkyl linker and a reverse transcription region. In some embodiments, the one or more oligonucleotides includes a PEG linker and a reverse transcription region. In some embodiments, the one or more oligonucleotides includes a targeting region and a reverse transcription region. In some embodiments, the one or more oligonucleotides includes a targeting region, an alkyl linker, and a reverse transcription region. In some embodiments, the oligo includes a targeting region, a polyethylene glycol (PEG) linker, and a reverse transcription region.

In some embodiments, the one or more oligonucleotides further includes a 5′ biotin. In some embodiments, the one or more oligonucleotides further includes a 3′ biotin. In some embodiments, the one or more oligonucleotides further includes a 5′ azide. In some embodiments, the one or more oligonucleotides further includes a 3′ azide. In some embodiments, the one or more oligonucleotides further includes a 5′ alkyne. In some embodiments, the one or more oligonucleotides further includes a 3′ alkyne. In some embodiments, the one or more oligonucleotides further includes a 5′ phosphate. In some embodiments, the one or more oligonucleotides further includes a 3′ phosphate. In some embodiments, the one or more oligonucleotides may include one or more of the foregoing.

In some embodiments, the method may further include measuring mRNA concentration. In some embodiments, the method may further include measuring total RNA concentration. In some embodiments, the method may further include fragmenting RNA. In some embodiments, the method may further include treating RNA with an RNase. In some embodiments, the method may further include measuring a DNA concentration. In some embodiments, the method may further include fragmenting DNA. In some embodiments, the method may further include treating DNA with an DNase.

In some embodiments, the method may further include a reverse transcription of the RNA sample. In some embodiments, the method may further include repairing cDNA ends. In some embodiments, the method may further include a cDNA sample bead cleanup step. In some embodiments, the method may further include cDNA sample quantification by qPCR step. In some embodiments, the method may include PCR amplification of cDNA and dual index addition.

In some embodiments, the targeting region of the one or more oligonucleotides may be complementary to the polyA tail. In some embodiments, the targeting region of the one or more oligonucleotides is complementary to a gene of interest.

In some embodiments, the method may include combining multiple oligonucleotides in the same sample to form a multiplexed mixture. In some embodiments, each oligonucleotide includes a unique barcode sequence. Through data analysis, if the sequences of barcode are known, the individual targeting oligonucleotides can be assigned from a mixed sample. Individual DNA or RNA molecules can then be attributed to each targeting oligo through the chimeric read structure of the resulting chimeric DNA or RNA formed by the barcode and the target DNA or RNA. In some embodiments, a barcode region may include one or more barcodes.

In some embodiments, preparing the DNA or RNA from a sample includes isolating cells. In some embodiments, the DNA or RNA from a sample may be taken from cells or tissue. Some embodiments further include lysing cells prior to isolating the complexes formed from the DNA or RNA containing a modification and an antibody. During the lysing process, cells may be incubated with lysis buffer and sonicated. In some embodiments, the lysing process further includes using RNase, such as RNase I, to partially fragment RNA molecules. In some embodiments, the lysing process further includes using DNase, such as DNase I, to partially fragment DNA molecules. In some embodiments, preparing the DNA or RNA from a sample includes isolating DNA or RNA from cell lysate. In some embodiments, preparing the DNA or RNA from a sample includes a sample from a viral source.

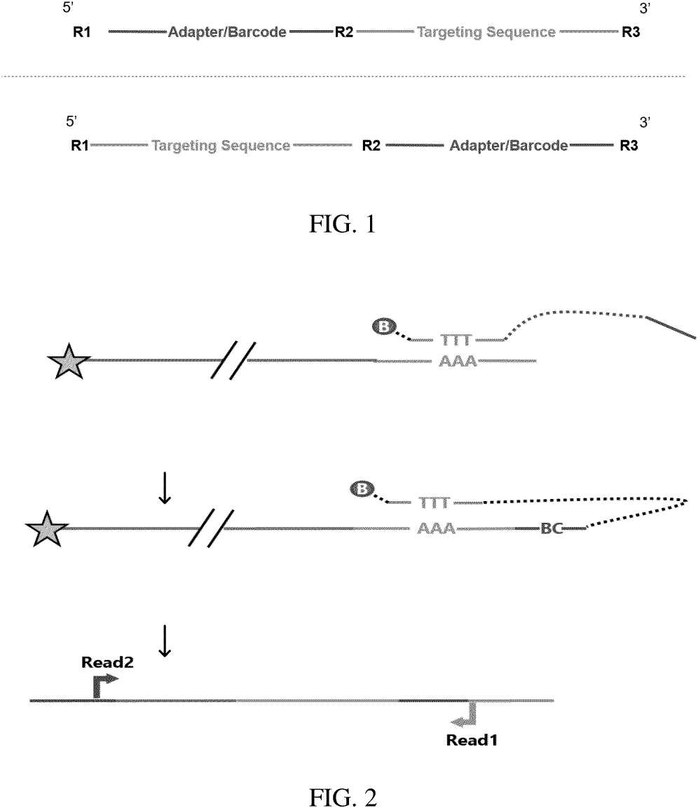

In some embodiments, isolating the DNA or RNA is done by precipitation. In some embodiments, the precipitation may include incubating the DNA, RNA or lysed cells with magnetic beads, which are pre-coupled to the targeting oligonucleotides (see FIG. 1). In some embodiments, using a magnet, the beads along with the DNA or RNA modification can be separated from the mix. In some embodiments, preparing the library includes coupling the targeting oligo to a magnetic bead. In some embodiments, the preparing the library further includes a precipitation step. In some embodiments, the precipitation step includes a first precipitation wash. In some embodiments, the precipitation step includes a second precipitation wash. In some embodiments, the precipitation step includes DNA or RNA end repair. In some embodiments, preparing the library further includes barcode chimeric ligation. In some embodiments, preparing the library further includes proteinase digestion of samples. In some embodiments, preparing the library further includes a clean-up step and a concentration step.

Some embodiments further include precipitated RNA end repair. In some embodiments, after the RNA oligo duplex are isolated, oligo and its target RNA molecules are ligated together to form oligo-target RNA chimeric molecules. Some embodiments further include repairing RNA ends using FastAP, a phosphatase that removes 5′-phosphate from RNA-DNA chimeric molecules, and/or T4 PNK, which converts 2′-3′-cyclic phosphate to 3′-OH that is needed for further ligation. In some embodiments, the method may further include the addition of a unique molecular identifier (UMI) and/or randomer into the antibody conjugated oligo to facilitate further processes. In some embodiments, the UMI may be a PCR duplicate removal. Some embodiments further include precipitated DNA end repair. In some embodiments, after the DNA oligo duplex is isolated, the oligonucleotide and its target DNA molecules are ligated together to form oligo-target DNA chimeric molecules. Some embodiments further include repairing DNA ends using FastAP, a phosphatase that removes a 5′-phosphate from RNA-DNA chimeric molecules, and/or T4 PNK, which converts 2′-3′-cyclic phosphate to 3′-OH that is needed for further ligation. In some embodiments, the method may further include the addition of a unique molecular identifier (UMI) and/or randomer into the antibody conjugated oligo to facilitate further processes. In some embodiments, the UMI may be a PCR duplicate removal.

In some embodiments, the RNA molecules may be incubated with proteases to digest the streptavidin and release the ligated RNA fragments from the precipitation beads. In some embodiments, the DNA molecules may be incubated with proteases to digest the streptavidin and release the ligated DNA fragments from the precipitation beads.

In some embodiments, the targeting oligonucleotides may be RNA, single stranded DNA (ssDNA), or synthetic nucleic acids, such as a locked nucleic acid (LNA). An LNA is often referred to as inaccessible RNA and is a modified RNA nucleotide in which the ribose moiety is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired and hybridize with DNA or RNA according to Watson-Crick base-pairing rules. The locked ribose conformation enhances base stacking and backbone preorganization. In some embodiments, this may significantly increase the hybridization properties (e.g., melting temperature) of oligonucleotides.

FIG. 1 illustrates an embodiment of two oligo designs for a proximity-based sequence specific ligation. In one embodiment, of the R1-Adapater/Barcode-R2-Targeting Sequence-R3, R1 includes a 5′-phosphate or a 5′-hydroxyl, R2 is either empty, a linker as described herein, a PEG linker, or an alkyl linker, and R3 includes one or more of the following biotin, azide, alkyne, PEG linker, and an alkyl linker. In one embodiment of the R1-Targeting Sequence-R2-Adapter/Barcord-R3, R1 includes one or more of the following biotin, azide, alkyne, PEG linker, alkyl linker, R2 is either empty, a linker as described herein, a PEG linker, or an alkyl linker, and R3 includes a 3′-phosphate or a 3′hydroxyl.

In one embodiment of the method described herein, RNA is isolated from a sample. The RNA may further undergo optional fragmentation. The hybridize sequence specific probe may be prebound to magnetic beads through biotin (designated as B in FIG. 2) interaction. Proximity ligation may further be performed. The RNA may be reverse transcribed using RT primer that anneals to the barcode (designated as BC on FIG. 2) region. The read adapter may be ligated to cDNA and amplified with indexing primers for sequencing.

### Kits

Also provided by this disclosure are kits for practicing the methods as described herein. For example, the kit may contain one or more targeted oligonucleotides. In some embodiments, the kit may include ligase. In some embodiments, the kit may include one or more buffers and reagents. In some embodiments, the kit may include ssDNA Adapter. The ssDNA Adapter may include ABCi7primer, DMSO, and bead elution buffer. In some embodiments, the kit may include an RT Adapter. In some embodiments, the kit may include one or more RT primers. The RT Adapter may include dNTPs and an ABC RT Primer. In some embodiments, the kit may include a bead elution buffer. The bead elution buffer may include TWEEN^{®} 20, Tris buffer, and EDTA. In some embodiments, the kit may include library elution buffer. The library elution buffer may include Tris buffer, EDTA and sodium chloride. In some embodiments, the kit may include qPCR primers. In some embodiments, the kit may include PNK buffer. The PNK buffer may include Tris buffer, magnesium chloride, and ATP. In some embodiments, the kit may include an RT buffer. In some embodiments, the RT buffer includes SuperScript III RT buffer and DTT. In some embodiments, the kit may include a proteinase K buffer. The proteinase K buffer may include Tris buffer, sodium chloride, EDTA, and SDS. In some embodiments, the kit may include bead binding buffer. The bead binding buffer may include RLT buffer and TWEEN^{®} 20. In some embodiments, the kit may include an RNA ligation buffer. The RNA ligation buffer may include Tris buffer, magnesium chloride, DMSO, TWEEN^{®} 20, ATP, and PEG. In some embodiments, the kit may include a no salt buffer. The no salt buffer may include Tris buffer, magnesium chloride, TWEEN^{®} 20, and sodium chloride. In some embodiments, the kit may include lysis buffer. The lysis buffer may include Tris buffer, sodium chloride, Igepal, SDS, and sodium deoxycholate. In some embodiments, the kit may include ssDNA ligation buffer. The ssDNA ligation buffer may include Tris buffer, magniesum chloride, DMSO, DTT, TWEEN^{®} 20, ATP and PEG8000. In some embodiments, the kit may include a high salt buffer. The high salt buffer may include Tris buffer, sodium chloride, EDTA, Igepal, SDS, and sodium deoxycholate. In some embodiments, the kit may include a coupling buffer. The coupling buffer may include Tris buffer, sodium chloride, EDTA, EGTA, NP-40, and TWEEN^{®} 20. In some embodiments, the kit may include a mRNA Elution buffer. The mRNA Elution buffer may include Tris buffer and EDTA. In some embodiments, the kit may include a 2x Hybridization buffer. The 2x Hybridization buffer may include Tris buffer, lithium chloride, TWEEN^{®} 20, and EDTA.

### EXAMPLES

Examples are provided herein below. However, the presently disclosed and claimed inventive concepts are to be understood to not be limited in their application to the specific experimentation, results and laboratory procedures. Rather, the Examples are simply provided as one of various embodiments and are meant to be exemplary, not exhaustive.

### Example 1

This example is a protocol for an oligo targeted proximity-based ligation experiment according to an embodiment of the disclosure.

### Buffer Compositions and Reagents

ssDNA Adapter: 50 uL 100 uM ABCi7primer, 60 uL DMSO, 140 uL Bead Elution Buffer
RT Adapter: 100 uL 10 mM dNTPs, 10 ul 10 uM ABC RT Primer
Bead Elution Buffer: 0.001% TWEEN^{®} 20, 10 mM Tris pH 7.5, 0.1 mM EDTA
Library Elution Buffer: 20 mM Tris pH 7.5, 0.2 mM EDTA, 5 mM NaCl
qPCR Primers: 1.25 mM Primer 1, 1.25 mM Primer 2
RT Primer: 6.7 mM each dNTP, 3.3uM ABC RT Primer
PNK Buffer: 97.2 mM Tris pH 7, 13.9 mM MgCl2, 1 mM ATP
RT Buffer: 2.17x SuperScript IIIRT buffer, 10 mM DTT
Proteinase K Buffer: 100 mM Tris pH 7.5, 50 mM NaCl, 10 mM EDTA, 0.2% SDS
Bead Binding Buffer: 1X RLT buffer, 0.01% TWEEN^{®} 20
RNA Ligation Buffer: 75 mM Tris pH 7.5, 16.7 mM MgCl2, 5% DMSO, 0.00067% TWEEN^{®} 20, 1.67 mM ATP, 25.7% PEG8000
ssDNA Ligation Buffer: 76.9 mM Tris pH 7.5, 15.4 mM MgCl2, 3% DMSO, 30.8 mM DTT, 0.06% TWEEN^{®} 20, 1.5 mM ATP, 27.7% PEG8000
Coupling buffer: 25 mM Tris pH 7.4, 125 mM NaCl, 1.25 mM EDTA, 1.25 mM EGTA, 0.125% NP-40, 0.125% TWEEN^{®} 20
mRNA Elution Buffer: 20 mM Tris pH 8, 1 mM EDTA
2x Hybridization Buffer: 50 mM Tris pH 7.4, 1 M LiCl, 0.2% TWEEN^{®} 20, 2.5 mM EDTA

TABLE 1

| Target | Barcode Sequence | SEQ ID NO |
|---|---|---|
| PolyA | /5Phos/NNN NNN NNT CTC GCG CAG ATC GGA AGA GCG TCG TGT/iSp18/TTT TTT TTT TTT TTT TTT /3Bio/ | 1 |
| m6A* | /5Phos/NNNNNCGATGAGATCGGAAGAGCGTCGTGT/3AmMO/ | 2 |
| m7G** | /5Phos/NNNNNTTAGGAGATCGGAAGAGCGTCGTGT/3AmMO/ | 3 |
| m7G*** | /5Phos/NNNNNTGACCAGATCGGAAGAGCGTCGTGT/3AmMO/ | 4 |

* Vendor Millipore Sigma and Cat # ABE572
** Vendor MBL and Cat # RN016
*** Vendor SYSY and Cat # 201001
3AmMO is a 3′ Amino Modifer modification iSp18 is an internal spacer 18 modification

TABLE 2

| Oligo | Sequence | SEQ ID NO |
|---|---|---|
| ABC RT Primer | ACACGACGCTCTTCC | 5 |
| ABC i7 Primer | /5Phos/AGATCGGAAGAGCACACGTCTG/3SpC3 | 6 |
| Index Primer 501 | AATGATACGGCGACCACCGAGATCTACACTATAGCCTACAC TCTTTCCCTACACGACGCTCTTCCGATCT | 7 |
| Index Primer 701 | CAAGCAGAAGACGGCATACGAGATCGAGTAATGTGACTGGA GTTCAGACGTGTGCTCTTCCGATCT | 8 |

3SpC3 is a 3′ C3 spacer modification

TABLE 3

| | | |
|---|---|---|
| Vendor | Part # | Reagent |
| New England Biolabs^{®} | M0331B | 5′ Deadenylase |
| New England Biolabs^{®} | M0544B | NEB NEXT^{®} Ultra™ II Q5^{®} Master Mix |
| New England Biolabs^{®} | P8107B | Proteinase K, Molecular Biology Grade |
| New England Biolabs^{®} | M0314B | RNase Inhibitor, Murine |
| New England Biolabs^{®} | M0201B | T4PNK |
| New England Biolabs^{®} | M0437B-BM | T4 RNA Ligase 1 (ssRNA Ligase) |
| ThermoFisher | EP1756B012 | SuperScript™ III Reverse Transcriptase Suppled with: 5X First-Strand Buffer - 1× 20 ml. Concentration 200 U/µl Pack Size 200 KU |
| Cell Signal | 9006 | ChIP-Grade Protein G Magnetic beads |
| ThermoFisher | AM2239B001 | TURBO™ DNase Supplied with: 10X TURBO™ DNase Buffer - 7× 1.85 ml |
| ThermoFisher | EF0652B001 | FastAP Thermosensitive AP Supplied with: 10X Buffer for fastAP - 10 × 1.5 ml |
| ThermoFisher | 37005D | Dynabeads™ MyOne™ Silane |
| Click Chemistry Tools | A134-10 | DBCO-PEG4-NHS Ester (10 mg) |
| Click Chemistry Tools | 1251-5 | 6-Azidohexanoic Acid Sulfo-NHS Ester (5 mg) |
| ThermoFisher | 89882 | Zebra™ Spin Desalting Columns, 7 K MWCO, 0.5 ml |
| Corning | 21-040-CV | PBS |
| ThermoFisher | AM2295 | RNase 1 |
| ThermoFisher | 75001.10.ml | ExoSAP-IT™ Express PCR Product Cleanup Reagent |
| ThermoFisher | 37050D | Oli(dT)25 |

### Prepare RNA

Total RNA was isolated from HEK293 cells using a Zymogen quick-RNA isolation kit (R1055) following manufactures protocol.

First, 50 µg of total RNA was transferred to a new 1.5 mL LoBind DNA tube. Second, if the volume of RNA was less than 200 µL; the volume was brought up to 200 µL using Molecular Biology Grade water. If RNA volume exceeded 200 µL, continued with volume and increased volume of 2× HyB when resuspending washed Oligo dT beads, so the final concentration of HyB is 1× during binding. Third, incubated RNA in a thermomixer for 2 minutes at 60° C. with interval mixing. Fourth, after incubation, immediately placed RNA samples on ice. Fifth, transferred 200 µL of Oligo dT beads into new 1.5 mL LoBind DNA tubes for each sample. Sixth, added 100 µL of 2× HyB to each tube containing 200 µL Oligo dT beads, and inverted the tube to mix. Seventh, placed the tube on DynaMag-2 magnet and allowed 1 minute for beads to separate. Eighth, slowly inverted the closed tubes on the magnet as beads started to separate to capture beads from the cap. Ninth, when the supernatant was transparent, the supernatant was discarded without disturbing beads. Tenth, removed the tube from the magnet and added 300 µL 2× HyB to each sample. Eleventh, inverted the tube to mix until homogeneous. Twelfth, placed the tube on DynaMag-2 magnet. Thirteenth, allowed 1 minute for the beads to separate. Fourteenth, slowly inverted the closed tubes on the magnet as the beads started to separate to capture the beads from the cap. Fifteenth, when the separation was complete, the supernatant was discarded without disturbing the beads. Sixteenth, repeated steps 6-15 for a total of two washes. Seventeenth, removed the tube from the magnet and added 200 µL of 2× HyB. Eighteenth, pipetted the mix to combine until homogeneous. Nineteenth, added entire volume (200 µL) of the beads in 2× HyB to 200 µL of denatured RNA (from step 4). Twentieth, placed the tube containing RNA and the beads on the tube rotator for 20 minutes at room temperature. Twenty-first, while the sample was rotating, diluted 2× HyB 5-fold according to Table 4. Twenty-two, placed the tube containing the beads and RNA on DynaMag-2 magnet. Twenty-three, allowed 1 minute for the beads to separate. Twenty-four, slowly inverted the closed tubes while on the magnet as the beads to separated to capture any beads from the cap. Twenty-five, when the separation was complete, the supernatant was discarded without disturbing the beads. Twenty-six, removed the tube from the magnet and added 745 µL of diluted HyB (Table 4).

TABLE 4

Dilution of 2× Hybridization Buffer (per sample)

| Component | Volume (µL) |
|---|---|
| 2x Hybridization Buffer (HyB) | 300 |
| Molecular Biology Grade water | 1200 |
| Total: | 1500 |

Twenty-seven, inverted the tube to mix until homogeneous. Twenty-eight, placed the tube on the DynaMag-2 magnet and allowed 1 minute for beads to separate. Twenty-nine, slowly inverted the closed tubes while on the magnet to capture any beads from the cap. Thirtieth, when the separation was complete, the supernatant was discarded without disturbing the beads. Thirty-one, spun the tube in the mini-centrifuge for 2 seconds. Thirty-two, the supernatant was discarded. Thirty-three, resuspended the beads in 200 µL mRNA Elution Buffer. Thirty-four, pipetted the mix to combine until homogeneous. Thirty-five, incubated the sample in the thermomixer for 2 minutes at 60° C. with interval mixing. Thirty-six, after incubation immediately placed on ice for 2 minutes. Thirty-seven, added 200 µL of 2× HyB into eluted the mRNA samples containing original oligo dT beads to have total volume of 400 µL. Thirty-eight, incubated on the tube rotator for 20 minutes at room temperature. Thirty-nine, once rotation was complete, placed the tube containing the beads and RNA on DynaMag-2 magnet. Fortieth, allowed 1 minute for the beads to separate. Forty-one, slowly inverted the closed tubes to separate and captured any beads from the cap. Forty-two, when separation was complete, the supernatant was discarded without disturbing the beads. Forty-three, removed the tube from magnet. Forty-four, added 745µL of diluted HyB (Table 4). Forty-five, inverted to mix the tube until homogeneous. Forty-six, placed tube on the DynaMag-2 magnet. Forty-seven, allowed 1 minute for the beads to separate. Forty-eight, slowly inverted the closed tubes to separate and captured any beads from the cap. Forty-nine, when separation was complete and the supernatant was transparent, aspirated and the supernatant was discarded without disturbing the beads. Fiftieth, spun the tube in the mini-centrifuge for 15 seconds. Fifty-one, aspirated all residual liquid. Fifty-second, added 40 µL Molecular Biology Grade water to bead the pellet. Fifty-third, pipetted the mix until homogeneous. Fifty-four, incubated the sample in thermomixer for 2 minutes at 60° C. with interval mixing. Fifty-five, magnetized immediately and transferred all supernatant to a new 1.5 mL LoBind DNA tube without disturbing the beads and placed on ice. Note: Volume pulled from beads was ~40 µL. Fifty-six, re-eluted sample a second time by adding 41 µL Molecular Biology Grade water to the beads. Fifty-seven, pipetted the mix until homogeneous. Fifty-eight, placed the sample in thermomixer set at 60° C. with interval mixing. Fifty-nine, increased the temperature to 70° C., allowed sample to transition temperatures. Sixtieth, incubated for a total of 3 minutes (starting from when temperature was increased) on thermomixer. Sixty-one, magnetized immediately and pooled all supernatant with the 1.5 mL LoBind DNA tube containing RNA (step 36) without disturbing beads. Note: Total volume of mRNA was around 80µL. Sixty-two, took ~80 samples into the following section (Measure mRNA Concentration).

### Measure mRNA Concentration

mRNA can be measured using a variety of methods. This protocol was optimized using Agilent 4200 TapeStation with Agilent’s High Sensitivity RNA ScreenTape, which measures both total RNA concentration and RNA integrity number. RIN is based on the ratio of 28S rRNA to 18S rRNA. Oligo dT beads selected out mRNA, so RIN was expected to be low due to the depletion of 28/18S rRNA, but the concentration of mRNA was still applicable. The expected mRNA yield was 1-3% of total RNA. For 50 µg starting RNA, 500 ng to 1.5 µg of final mRNA was expected.

### Optional Stopping Point: RNA Samples Stored at 80 ℃

### Next Stopping Point: 1 Hour

### Fragment mRNA

First, aliquoted 420 ng of eluted mRNA to new signed 0.2 mL PCR tube strip and prepared mRNA fragmentation mix for each sample according to Table 5.

TABLE 5

mRNA fragmentation Mix (per sample)

| Component | Volume (µL) |
|---|---|
| RNA + Molecular Biology Grade Water | 67 |
| Turbo DNase Buffer | 8 |
| RNase Inhibitor | 2 |
| Turbo DNase | 3 |
| Total: | 80 |

Second, mixed the sample well. Third, incubated samples in a PCR machine: 37° C. for 10 minutes, 95° C. for 16 minutes, and 5° C. for 10 sec, with lid at 98° C. Fourth, placed samples on ice or freeze them at -80° C. after incubation.

### Library Prep

### Preparation

Placed coupling buffer at 4° C.

### Procedure

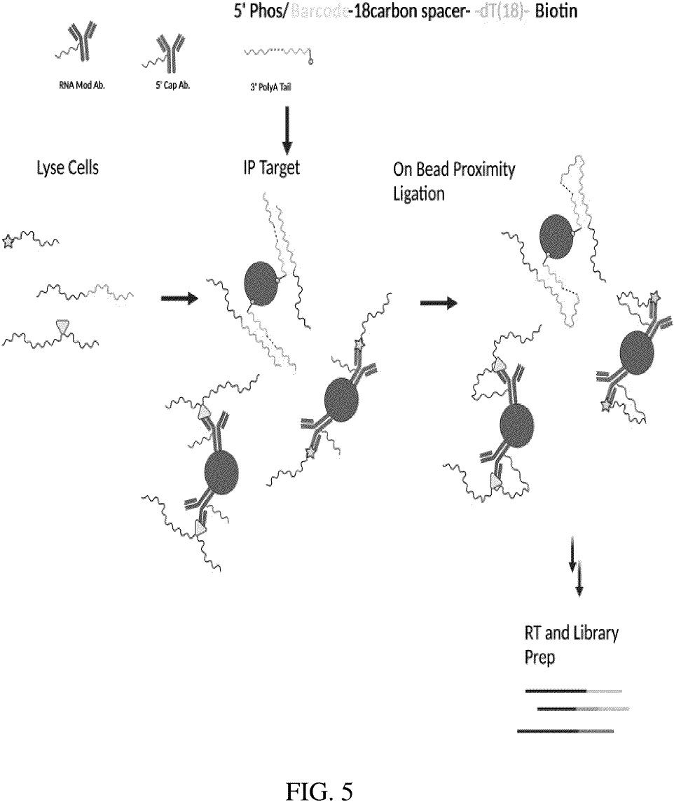

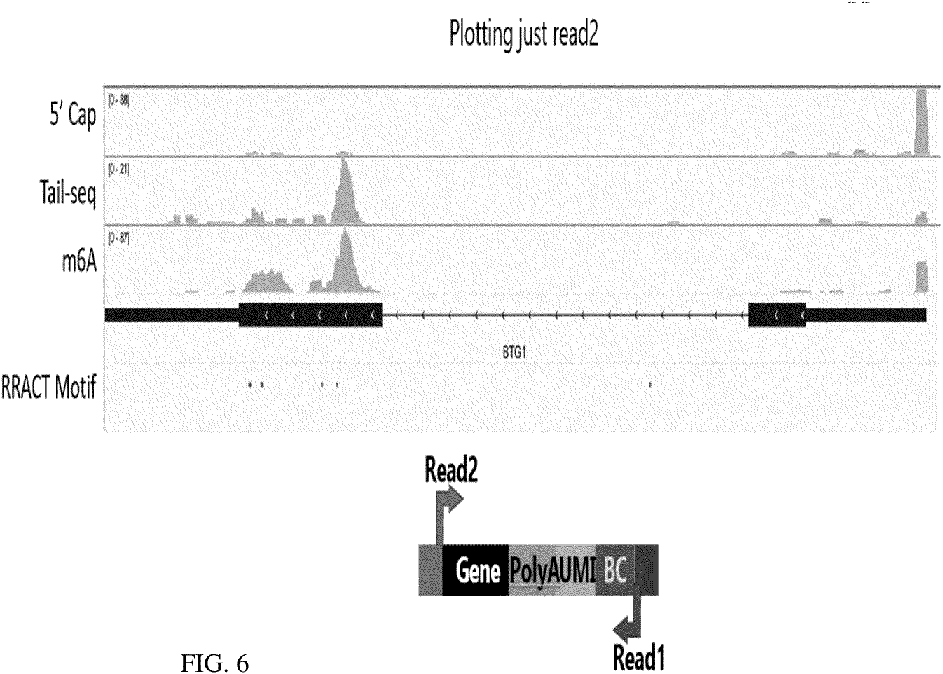

Coupling oligonucleotides to magnetic beads pre-coupled with secondary antibody. First, Dynabeads MyOne Streptavidin C1 magnetic beads were mixed until homogeneous. Second, transferred 25 µL Dynabeads MyOne Streptavidin C1 per sample into a fresh 1.5 mL LoBind tube (e.g., for 3 samples, use 75 µL of secondary beads). Third, added 200 µL of coupling buffer (chilled) to the tube with secondary beads. Fourth, placed the tube on DynaMag-2 magnet. Fifth, after separation was complete and supernatant was transparent (~ 1 minute), carefully aspirated and discarded the supernatant without disturbing the beads. Sixth, removed the tube from the magnet. Seventh, added 500 µL coupling buffer (chilled) to the tube, closed the tube and inverted the mix until homogeneous. Eighth, placed the tube on DynaMag-2 magnet. Ninth, after separation was complete and the supernatant was transparent (~ 1 minute), carefully aspirated and discarded the supernatant without disturbing the beads. Tenth, removed the tube from the magnet. Eleventh, repeated steps 7-10 once for a total of two washes. Twelfth, added 50 µL coupling buffer (chilled) per sample to the tube. Thirteenth, added 1 µL of 100 µM oligo per sample to the washed beads. Some of the samples also had 5 µg of primary antibody per sample added to the tube containing washed beads to provide a multiplexed assay with both oligo-bound and primary antibody-bound beads (such as shown in FIG. 5). Fourteenth, placed the tube on the tube rotator and allow the beads and biotin oligo to couple for 1 hour at room temperature.

### Precipitation

First, the oligo-coupled magnetic bead tubes were removed from the rotator. Second, to each oligo-coupled magnetic bead tube, 500 µL coupling buffer (chilled) was added. Third, inverted the mix until homogenous. Fourth, placed the tubes on DynaMag-2 magnet to separate the beads and allowed at least 1 minute for bead separation. Fifth, when separation was complete, and liquid was transparent, carefully aspirated and discarded supernatant without disturbing the beads. Sixth, repeated steps 2-5 for a total of 2 washes. Seventh, removed tubes from magnet. Eighth, added 50 µL coupling buffer (chilled) per sample to the tubes. Ninth, used 50 µL of oligo coated bead to 50 ng of fragmented RNA in 250 µL coupling buffer and slowly pipetted to mix until homogeneous. Tenth, rotated precipitation tubes containing fragmented RNA, and antibody-coupled magnetic beads overnight at 4° C.

### Stopping Point: Samples Rotate Overnight at 4° C. for Up to 16 Hours

### Preparation

Prewarm Thermomixer to 37° C.

### Procedure

### First Precipitation Wash

First, placed precipitation tubes on DynaMag-2 magnet to separate the beads. Second, allowed at least 1 minute for bead separation. Third, when the separation was complete and liquid was transparent, carefully aspirated and discarded the supernatant without disturbing the beads. Fourth, removed precipitation tubes from the magnet. Fifth, added 500 µL cold coupling buffer. Sixth, inverted the mix until homogeneous. Seventh, placed the mix on the DynaMag-2 magnet. Eighth, while on the magnet, slowly inverted the closed tubes as the beads started to separate to capture any beads from the cap. Ninth, when separation was complete, and liquid was transparent, gently opened the tubes and discarded the supernatant without disturbing the beads. Tenth, removed precipitation tubes from magnet and added 100 µL cold coupling buffer. Eleventh, closed tubes well and vortexed for 15 seconds. Twelfth, incubated on the tube rotator for 3 min at room temperature. Thirteenth, placed the tube on the DynaMag-2 magnet. Fourteenth, while on the magnet, slowly inverted the closed tubes as the beads start to separate to capture any beads from the cap. Fifteenth, when the separation was complete, and the liquid was transparent, the tubes were gently opened and the supernatant was discarded without disturbing the beads. Sixteenth, repeated steps 5-9 for times for a total of two washes. Seventeenth, removed precipitation tubes from magnet. Eighteenth, added 100 µL cold coupling buffer to the tube. Nineteenth, inverted mix until homogenous. Twentieth, placed samples on ice and proceed immediately to the next step.

### RNA End Repair

First, the PNK Enzyme master mix was prepared according to Table 6 below in a fresh 1.5 mL LoBind tube. Note: Include 3% excess volume to correct for pipetting losses.

TABLE 6

PNK Enzyme master mix (per sample)

| Reagent | Volume (µL) |
|---|---|
| PNK Buffer | 76 |
| RNase Inhibitor | 1 |
| PNK Enzyme | 3 |
| Total: | 80 |

Second, moved all IP tubes from ice to DynaMag-2 magnet and allowed at least 1 minute for bead separation. Third, removed and discarded supernatant. Forth, spin all samples in mini-centrifuge for 3 seconds. Fifth, place samples back on magnet and allow 1 minute for bead separation. Sixth, pipetted and discarded any excess liquid without disturbing beads. Seventh, added 80 µL of PNK Enzyme master mix to each IP tube. Pipette to mix. Eighth, incubated in thermomixer at 37° C. for 20 minutes with interval mixing at 1,200 rpm.

### Second Precipitation Wash

First, when the precipitation RNA end repair was complete, removed tubes from the Thermomixer and added 500 µL cold coupling buffer directly to the samples. Second, inverted the mix until homogeneous. Third, placed the precipitation tubes on the DynaMag-2 magnet to separate beads. Fourth, allowed at least 1 minute for the bead separation. Fifth, when separation was complete and liquid was transparent, carefully aspirated and discarded supernatant without disturbing the beads. Sixth, removed precipitation tubes from the magnet. Seventh, added 250 µL cold coupling buffer. Eighth, inverted the mix until homogeneous. Ninth, separated the beads on the magnet and removed the supernatant without disturbing the beads. Tenth, removed the precipitation tubes from the magnet. Eleventh, added 250 µL cold coupling buffer. Twelfth, inverted the mix until homogeneous. Thirteenth, separated the beads on the magnet and removed the supernatant without disturbing the beads. Fourteenth, spun all precipitation samples in the mini-centrifuge for 3 seconds. Fifteenth, placed the samples back on the magnet and allowed 1 minute for the bead separation. Sixteenth, pipetted and discarded excess liquid without disturbing the beads. Seventeenth, removed precipitation tubes from the magnet. Eighteenth, added 250 µL cold coupling buffer. Nineteenth, inverted the mix until homogeneous. Twentieth, placed the samples on ice and proceeded immediately to the next step.

### Barcode Chimeric Ligation

First, the Chimeric Ligation master mix was prepared according to Table 7 in a fresh 1.5 mL LoBind tube. Note: Included 3% excess volume to correct for pipetting losses. Note: RNA Ligation Buffer was very viscous, and the master mix required thorough mixing.

TABLE 7

Chimeric Ligation master mix (per sample)

| Reagent | Volume (µL) |
|---|---|
| Molecular Biology Grade Water | 43 |
| RNA Ligation Buffer | 94 |
| RNase Inhibitor | 2 |
| T4 Ligase | 11 |
| Total: | 150 |

Second, moved all the precipitation tubes from ice to the DynaMag-2 magnet and allowed at least 1 minute for the bead separation. Third, removed and discarded the supernatant. Fourth, spun all the samples in the mini-centrifuge for 3 seconds. Fifth, placed the samples back on the magnet and allowed 1 minute for the bead separation. Sixth, pipetted and discarded any excess liquid without disturbing the beads. Seventh, slowly added 150 µL of the Chimeric Ligation master mix to each precipitation tube. Gently pipetted the mix until homogenous. Eighth, placed the precipitation tubes on tube rotator for 45 minutes at room temperature. Ninth, separated the beads on the magnet and removed the supernatant without disturbing the beads. Tenth, removed precipitation tubes from the magnet. Eleventh, added 250 µL cold coupling buffer. Twelfth, inverted the mix until homogeneous. Thirteenth, separated the beads on the magnet and removed the supernatant without disturbing the beads. Fourteenth, added 250 µL cold coupling buffer. Fifteenth, inverted the mix until homogeneous. Sixteenth, separated the beads on the magnet and removed the supernatant without disturbing the beads. Seventeenth, repeated steps 12-14 for a total of 2 washes.

### Proteinase Digestion of Samples

First, the proteinase master mix was prepared according to Table 8 below in a fresh 1.5 mL LoBind tube. Note: Included 3% excess volume to correct for pipetting losses.

TABLE 8

Proteinase master mix (per sample)

| Reagent | Volume (µL) |
|---|---|
| Proteinase Buffer | 110 |
| Proteinase Enzyme | 17 |
| Total: | 127 |

Second, added 127 µL of the Proteinase master mix to each sample tube containing IP beads and ensured all the beads were submerged. Third, incubated in the thermomixer at 37° C. for 20 minutes with interval mixing at 1,200 rpm. Fourth, after completion of the first incubation, increased the temperature to 50° C. and continued the incubation in the thermomixer at 50° C. for 20 minutes with interval mixing at 1,200 rpm.

### Clean All Samples With Zymo RNA Clean & Concentrator Kit

Preparative Note: Ensured 100% EtOH was added to the RNA Wash Buffer concentrate upon the first usage. Preparative Note: Centrifugation steps were done at room temperature.

First, for each sample, all liquid (~125 µL) was transferred from proteinase digestion into fresh, labeled DNA LoBind tubes. This contained the eluted RNA sample. Second, added 250 µL of the RNA Binding Buffer to the 125 µL of the eluted RNA sample. Pipetted to mix. Third, added 375 µL of 100% ethanol to the tubes. Fourth, pipetted the mix thoroughly. Fifth, transferred all the liquid (750 µL) to corresponding labeled filter-columns in collection tubes. Sixth, centrifuged at 7,000 x g for 30 seconds. Discarded flow-through. Seventh, added 400 µL of RNA Prep Buffer to each column. Eighth, centrifuged at 7,000 x g for 30 seconds. Discarded the flow-through. Ninth, added 480 µL of the RNA Wash Buffer to each column. Tenth, centrifuged at 7,000 x g for 30 seconds. Discarded the flow-through. Eleventh, repeated the steps 10-11 once for a total of 2 washes. Twelfth, laced each spin column in a new collection tube. Discarded used collection tubes. Thirteenth, ‘Dry’ spun at 10,000 x g for 1 minute to remove any residual ethanol. Fourteenth, transferred each filter-column to a new labeled 1.5 mL LoBind tube. Discarded the used collection tubes. Fifteenth, opened the columns’ caps and allowed to air dry for 3 minutes. Sixteenth, eluted all the samples by adding 11 µL of Molecular Biology Grade Water directly to each filter. Seventeenth, incubated at room temperature for 1 minute. Eighteenth, centrifuged at 12,000 x g for 90 seconds. Discarded filter-columns. Note: Elution volume was ~10 µL. Nineteenth, if proceeding to next step, store all samples on ice. Precipitation samples can remain on ice or be frozen until Reverse Transcription and cDNA Adapter Ligation.

### Optional Stopping Point: If Stopping Here, RNA Samples Should Be Stored at -80° C.

### Next Stopping Point: ~2h

### Reverse Transcription of Sample Reagent Preparation

First, for each precipitation RNA sample, 9 µL was transferred into a new, labeled 0.2 mL strip tube. Second, added 1.5 µL of the RT Primer into RNA. Third, mixed, and spun all the samples in the mini-centrifuge for 5 seconds to draw all liquid to the bottom of the tube. Fourth, incubated at 65° C. for 2 minutes in the thermal cycler with the lid preheated to 70° C. Fifth, after incubation, immediately transferred to the ice for 1 minute. Sixth, adjusted the thermal cycler block temperature to 54° C. - 20 minutes (with the lid set to 65° C.).

### Reverse Transcription of RNA

First, the Reverse Transcription Master Mix was prepared according to Table 9 in a fresh 1.5 mL LoBind tube. Second, pipette the sample up and down 10 times to mix. Third, stored the samples on ice until used. Note: Included 3% excess volume to correct for pipetting losses.

TABLE 9

Reverse Transcription Master-Mix (per sample)

| Component | Volume (µL) |
|---|---|
| RT Buffer | 9.2 |
| RNase Inhibitor | 0.2 |
| Superscript III | 0.6 |
| Total: | 10 |

Fourth, added 10 µL of the Reverse Transcription Master Mix to each sample, leaving the samples on ice. Pipetted to mix. Fifth, spun samples in mini-centrifuge for 5 seconds to draw all liquid to the bottom of the tube. Sixth, immediately incubated samples at 54° C. for 20 minutes in the thermal cycler with the lid at 65° C. Seventh, after incubation, immediately placed samples on ice. Eighth, adjusted the thermal cycler block temperature to 37° C. - 15 minutes (with the lid set to 45° C.).

### cDNA End Repair of Samples

First, 2.5 µL of the ExoSap-IT was added to each sample. Second, spun the samples in the mini-centrifuge for 5 seconds to draw all the liquid to the bottom of the tube. Third, incubated in the thermal cycler at 37° C. for 15 minutes with the lid at 45° C. Fourth, removed the strip-tube and placed the samples on ice. Fifth, adjusted the thermal cycler block to 70° C. - 10 minutes (with lid set to 75° C.). Sixth, added 1 µL of 0.5 M EDTA (pH 8) to each sample. Seventh, pipetted the samples up and down gently 5 times to mix. Eighth, added 3 µL of 1 M NaOH to each sample. Ninth, pipetted the samples up and down gently 5 times to the mix. Tenth, incubated the tubes at 70° C. for 10 minutes in the thermal cycler with the lid at 75° C. Eleventh, placed the strip-tube on ice for 10 seconds. Twelfth, added 3 µL of 1 M HCl to each sample. Thirteenth, proceeded immediately to the next step.

### cDNA Sample Bead Cleanup

Preparation Note: Thawed ssDNA Adapter and ssDNA Ligation Buffer at room temperature until completely melted, then stored ssDNA Adapter on ice and ssDNA Ligation Buffer at room temperature. Preparation Note: Prepared fresh 80% Ethanol in Molecular Biology Grade water in a fresh 50 mL tube if was not done previously. Stored at room temperature for up to 1 week. Kept the tube closed tightly.

First, the Silane beads (provided) out of 4° C. were taken and resuspended until homogeneous. Second, washed the Silane beads prior to addition to the samples. Third, for each cDNA sample, transferred 5 µL of the Silane beads to a new 1.5 mL DNA LoBind tube (e.g., for 4 samples, transferred 20 µL of Silane beads). Fourth, added 5× volume of the Bead Binding Buffer (e.g. for 4 samples add 100 µL buffer to 20 µL of Silane beads). Pipetted up and down to mix until the sample was homogeneous. Fifth, placed the tube on the DynaMag-2 magnet. When the separation was complete and the supernatant was clear, carefully aspirated and discarded the supernatant without disturbing the beads. Sixth, removed the tube from the magnet. Seventh, resuspended the Silane beads in 93 µL of the Bead Binding Buffer per sample. Eighth, pipetted up and down until the beads are fully resuspended. Ninth, added 90 µL of washed Silane beads to each cDNA sample. Tenth, pipetted up and down to mix until sample is homogeneous. Eleventh, added 90 µL of 100% EtOH to each cDNA sample. Twelfth, pipetted the mix until homogeneous. Thirteenth, incubated at room temperature for 10 minutes, with pipette mixing every 5 minutes. Fourteenth, moved the samples to fresh strip tube: placed a new, labeled 0.2 mL strip tube on the 96-well magnet and transferred the sample from old to new strip tube. Fifteenth, allowed to incubate for 1 minute or until separation was completed and the liquid was transparent. Sixteenth, carefully discarded the supernatant without disturbing the beads. Seventeenth, added 150 µL of 80% EtOH, Eighteenth, moved the samples to different positions on the magnet to wash thoroughly. Nineteenth, added an additional 150 µL of 80% EtOH. Twentieth, incubated on the magnet for 30 seconds until separation was completed and the supernatant was transparent. Twenty-first, carefully aspirated and discarded all the supernatant while on the magnet. Twenty-second, repeated steps 17-21 once for a total of two washes. Twenty-third, capped the samples were spun in the mini-centrifuge for 5 seconds to draw all the liquid to the bottom of the tube. Twenty-fourth, placed the tube back on 96-well magnet. Twenty-fifth, incubated on the magnet for 10 seconds until the separation was complete and the supernatant was transparent. Twenty-sixth, using fine tips, aspirated and discarded all the residual liquid without disturbing beads while on magnet. Twenty-seventh, allowed the beads to air dry for 5 minutes or until beads no longer appeared wet and shiny. Note: Do not over dry samples. Twenty-eighth, once completely dry, carefully removed the tubes from the magnet. Twenty-ninth, resuspended the beads in 2.5 µL of the ssDNA Adapter. Thirtieth, pipetted to mix until homogeneous. Thirty-first, incubated in the thermal cycler at 70° C. for 2 minutes with the lid at 75° C. Thirty-second, following incubation, immediately place on ice for 1 minute.

### cDNA Ligation on Beads

First, the cDNA Ligation master mix was prepared according to Table 10 in a fresh 1.5 mL LoBind tube. Pipetted the mix to combine (do not vortex). Used immediately. Note: Include 3% excess volume to correct for pipetting losses.

TABLE 10

cDNA Ligation Master Mix (per sample)

| Component | Volume (µL) |
|---|---|
| ssDNA Ligation Buffer | 6.5 |
| T4 Ligase | 1 |
| Deadenylase | 0.3 |
| Total: | 7.8 |

Second, 7.8 µL of cDNA Ligation master mix was slowly added to each sample from the previous section cDNA Bead Clean Up) and pipetted the mix until homogeneous. Third, incubated at room temperature overnight on a tube rotator.

### Procedure

### Ligated cDNA Sample Cleanup

First, ligated-cDNA samples from the tube rotator was obtained. Second, to each cDNA sample, added 5 µL of the Bead Elution Buffer. Third, added 45 µL of the Bead Binding Buffer. Pipetted to mix. Fourth, added 45 µL of 100% EtOH to each sample and pipetted the mix until homogeneous. Fifth, incubated at room temperature for 10 minutes, with pipette mixing every 5 minutes. Sixth, placed the strip-tube on the 96-well magnet and allowed to incubate for 1 minute or until separation was complete and liquid was transparent. Seventh, carefully aspirated and discarded the supernatant without disturbing the beads. Eighth, added 150 µL of 80% EtOH without disturbing the beads. Ninth, moved the samples to different positions on the magnet to wash thoroughly. Tenth, carefully added an additional 150 µL of 80% EtOH. Eleventh, incubated on the magnet for 30 seconds or until separation was complete and the supernatant was transparent. Twelfth, carefully aspirated and discarded the supernatant. Thirteenth, repeated steps 7-11 for a total of two washes. Fourteenth, spun the capped samples in the mini-centrifuge for 3 seconds to draw all liquid to the bottom of the tube. Fifteenth, placed the tube back on 96-well magnet. Sixteenth, incubated on the magnet for 30 seconds or until the separation was complete and the supernatant was transparent. Seventeenth, while on the magnet, aspirated and discarded all residual liquid without disturbing the beads. Eighteenth, allowed the beads to air dry for 5 minutes or until the beads no longer appear wet and shiny. Nineteenth, once completely dry, carefully removed the tubes from the magnet. Twentieth, added 25 µL the Bead Elution Buffer to each the sample. Twenty-one, pipetted up and down to mix until the sample was homogeneous. Twenty-two, incubated for 5 minutes at room temperature. Twenty-third, after incubation, moved the tubes to 96-well magnet. Twenty-fourth, incubated on the magnet for 30 seconds until the separation was complete and the supernatant was transparent. Twenty-fifth, transferred the supernatant (containing eluted cDNA) to new 0.2 mL strip tubes.

### Optional Stopping Point: If Stopping Here, Eluted cDNA Samples Should Be stored at -80° C.. Next stopping point: ~2 hrs

### cDNA Sample Quantification by qPCR

First, the qPCR master mix was prepared for the appropriate number of reactions according to Table 11 in a fresh 1.5 mL LoBind tube. Note: Include 3% excess volume to correct for pipetting losses.

TABLE 11

qPCR quantification master mix (per sample)

| Component | Volume (µL) |
|---|---|
| NEB LUNA Universal qPCR 2× Master Mix | 5 |
| qPCR Primers | 4 |
| Total: | 9 |

Second, obtained and labeled a 96- or 384-well qPCR reaction plate. Third, added 1 µL of eluted the cDNA samples to 9 µL of Molecular Biology Grade Water for a 1:10 dilution. Fourth, added 9 µL of the qPCR master mix into all assay wells on ice. Fifth, added 1 µL of each diluted cDNA (or water for negative controls) into the designated well. Note: Stored remaining diluted cDNA samples on ice until PCR in the next section. Sixth, covered the plate with a MicroAmp adhesive film and sealed with MicroAmp adhesive film applicator. Seventh, spun the plate at 3,000 × g for 1 minute. Eighth, the qPCR assay was run according to the user manual for the specific instrument. Ninth, ran parameters appropriate for SYBR. Note: For example, for the StepOnePlus qPCR system the appropriate program was: 95° C. -30 sec, (95° C. - 10 sec, 65° C. - 30 sec) × 32 cycles; No melting curve. Tenth, recorded qPCR Ct values for all samples. Eleventh, set threshold to 0.5 - this recommendation was for StepOnePlus System. Note: Typical acceptable Ct values range from 10 to 23. For robust estimation, Ct values for samples should be ≥ 10. If values are below 9, dilute the 1:10 diluted cDNA an additional 10-fold, and re-perform qPCR using the 1:100 diluted cDNA.

### PCR Amplification of cDNA and Dual Index Addition

First, the Index primers were thawed at room temperature until fully melted. Shook to mix and spun in mini-centrifuge for 3 seconds. Stored on ice until use. Second, prepared the PCR amplification reaction mix according to Table 12 in fresh 0.2 mL PCR strip-tubes. Kept tubes on ice. Note: If samples were going to be multiplexed during high-throughput sequencing, ensure that all samples to be pooled together have a unique combination of indexing primers.

TABLE 12

PCR amplification reaction mix contents (prepare individually for each sample) -Can use traditional Illumina index primers

| Component | Volume (µL) |
|---|---|
| Ligated cDNA | 16 |
| 501 Index Primer | 2 |
| 701 Index Primer | 2 |
| PCR mix | 20 |
| Total: | 40 |

Third, pipetted mix to combine. Fourth, spun samples in the mini-centrifuge for 3 seconds to draw all liquid to the bottom of the tube. Fifth, kept samples on ice. Sixth, referred to Ct values recorded to calculate the appropriate number of cycles for each sample. Used formula to calculate N = Ct - 6, where N is the number of cycles performed using the second (two-step) cycling conditions: N + 6 = Total cycles = Ct, TOTAL number of PCR cycles for final library amplification = 6+N. Note: e.g. If Ct = 13.1, then N = 7, and the Total number of PCR cycles equals 13 (6+7).

Seventh, PCR for the specific number of cycles calculated for each sample was run according to the PCR Amplification program shown in Table 13.

TABLE 13

PCR Amplification program

| Temperature | Time | Cycles |
|---|---|---|
| 98° C. | 30 seconds | |
| 98° C. | 15 seconds | 6 |
| 70° C. | 30 seconds | |
| 72° C. | 40 seconds | |
| Extra N cycles (N = Ct value - 9) | | |
| 98° C. | 15 seconds | N* |
| 72° C. | 45 seconds | |
| 72° C. | 1 minute | |
| 4° C. | ∞ | |

Total number of PCR cycles 6+N
*N should be ≥ 1 and < 14.

Eighth, samples were immediately placed on ice to cool following PCR amplification.

### AMPure Library PCR Product Cleanup

Preparative Note: Allowed the AMPure XP beads to equilibrate at room temperature for 5 minutes.

First, the AMPure XP beads were manually shaken or vortexed to resuspend the sample until homogeneous. Second, added 60 µL of the AMPure XP beads into each 40 µL PCR reaction. Third, pipetted to mix until the sample is homogeneous. Fourth, incubated at room temperature for 10 minutes, with pipette mixing every 5 minutes. Fifth, placed strip-tube on 96-well magnet, and allowed to incubate for 1 minute or until separation was complete and the liquid was transparent. Sixth, carefully aspirated and discarded the supernatant without disturbing the beads. Seventh, added 150 µL of 80% EtOH without disturbing the beads. Eighth, moved the samples to different positions on the magnet to wash thoroughly. Ninth, carefully added an additional 150 µL of 80% EtOH. Tenth, incubated on the magnet for 30 seconds or until the separation was complete and the supernatant was transparent. Eleventh, carefully aspirated and discarded the supernatant. Twelfth, repeated steps 7-11 for a total of two washes. Thirteenth, spun the capped samples in the mini-centrifuge for 3 seconds to draw all liquid to the bottom of the tube. Fourteenth, placed the tube back on 96-well magnet. Fifteenth, incubated on the magnet for 30 seconds or until the separation was complete and the supernatant was transparent. Sixteenth, while on the magnet, aspirated and discarded all the residual liquid without disturbing the beads. Seventeenth, allowed the beads to air dry for 5 minutes or until the beads no longer appeared wet and shiny. Eighteenth, once completely dry, carefully removed the tubes from magnet. Nineteenth, added 20 µL Molecular Biology Grade Water to each sample. Twentieth, pipetted the mix until sample is homogeneous. Twenty-first, incubated for 5 minutes at room temperature. Twenty-second, transferred the 20 µL of eluted sample to new strip-tube. Twenty-third, analyzed library length and concentration via Agilent Tapestation. Twenty-fourth, if adapter dimer was present, preform an agarose gel extraction for a DNA 200-400 nts in length and retapestation.

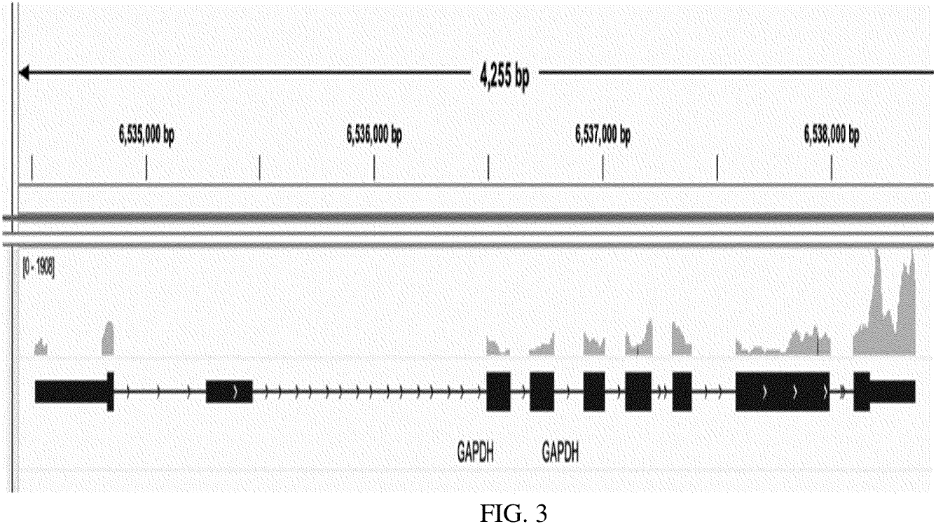

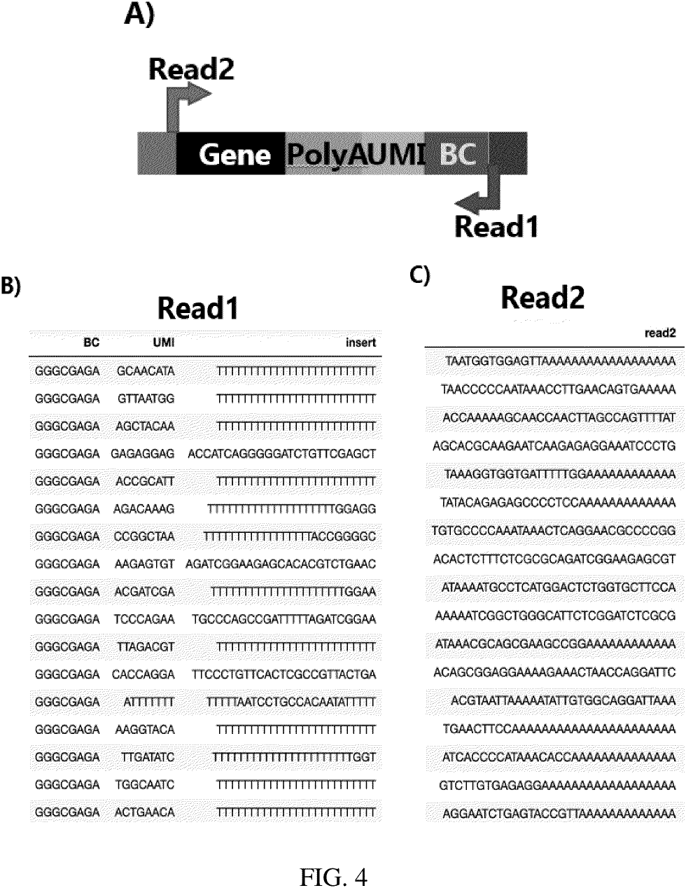

FIGS. 3-4 illustrates the results of the protocol described above. FIG. 3, illustrates a genome track view of one polyA tail enrichment using proximity-based polyT ligations and depicts 3′ coverage bias. FIG. 4, Panel A) illustrates the read structure of a polyA tail enrichment using proximity-based polyT ligations. Panel B) is a table of read 1 from a polyA tail enrichment and depicts sequencing of the barcode (BC), unique molecular index (UMI), and polyA tails (complementary strand) in the sequencing insert. Panel C) is a table of read 2 from a polyA tail enrichment and depicts sequencing of expressed genes within the sample with sequencing extending into the polyA tail.

What is claimed is:

1. A method of oligonucleotide targeted proximity ligation, the method comprising:
hybridizing a nucleic acid sample with one or more oligonucleotides, wherein the one or more oligonucleotides comprise a targeting region and an amplification region;
performing a proximity based ligation between the nucleic acid sample and one or more hybridized oligonucleotides; and
preparing a library of nucleic acids amplified from the one or more oligonucleotides.

2. The method of claim 1, wherein the one or more oligonucleotides comprises a targeting region, an alkyl linker, a reverse transcription region, a reverse transcription primer region, a barcode region, targeting region, a polyethylene glycol (PEG) linker, a reverse transcription region or a combination thereof.

3. The method of claim 1, wherein the one or more oligonucleotides further comprises a 5′ biotin, 3′ biotin, 5′ azide, 3′ azide, 5′ alkyne, 3′ alkyne, 5′ phosphate, or a combination thereof.

4. The method of claim 1, wherein preparing the nucleic acid sample further comprises isolating cells.

5. The method of claim 1, further comprising measuring nucleic acid concentration.

6. The method of claim 1, further comprising fragmenting the nucleic acid sample.

7. The method of claim 6, wherein fragmenting the nucleic acid sample is performed by the group consisting of heating the sample, treatment with nuclease, addition of metal ions, mechanical shearing, or a combination thereof.

8. The method of claim 2, wherein the barcode region comprises one or more barcodes.

9. The method of claim 1, wherein preparing the library comprises coupling the one or more oligonucleotides to a magnetic bead.

10. The method of claim 1, wherein preparing the library comprises a precipitation step.

11. The method of claim 10, wherein the precipitation step comprises a first precipitation wash, a nucleic acid end repair, a second precipitation wash, or a combination thereof.

12. The method of claim 1, wherein preparing the library further comprises barcode chimeric ligation, proteinase digestion of samples, a clean up step and a concentration step, a reverse transcription of the nucleic acid sample, or a combination thereof.

13. The method of claim 1, further comprising repairing DNA ends step, a DNA sample bead cleanup step, a DNA sample quantification by qPCR step, PCR amplification of DNA and dual index addition, or a combination thereof.

14. The method of claim 1, wherein the targeting region of the one or more oligonucleotides is complementary to a polyA tail.

15. The method of claim 1, wherein the targeting region of the one or more oligonucleotides is complementary to a gene of interest.

16. The method of claim 1, further comprising a clean up step.

17. The method of claim 16, wherein the clean up step removes protease and buffer from the library of nucleic acids.

18. The method of claim 16, wherein the clean up step proceeds a reverse transcription step of the preparing a library of nucleic acids.

19. A kit comprising:
one or more targeted oligonucleotides; and
a manual providing instructions for proximity based ligation enrichment.

20. The kit of claim 19, further comprising one or more buffers, wherein the one or more buffers is selected from the group consisting of bead elution buffer, library elution buffer, PNK buffer, RT buffer, proteinase K buffer, bead binding buffer, RNA ligation buffer, ssDNA ligation buffer, and coupling buffer.

21. The kit of claim 19, further comprising one or more primers, wherein the one or more primers is selected from the group consisting of qPCR primer and RT primer.
